(19) United States
(12) Patent Application Publication
(10) Pub. No.: US 2022/0347190 A1
(43) Pub. Date: Nov. 3, 2022

(54) TREATMENT COMPRISING FXR AGONISTS

(71) Applicant: Novartis AG, Basel (CH)

(72) Inventors: Michael Badman, Newton, MA (US); Clifford Brass, New York, NY (US); Bryan Laffitte, Cardiff, CA (US); Iwona KSIAZEK, Basel (CH)

(21) Appl. No.: 17/760,893

(22) PCT Filed: Sep. 18, 2020
(86) PCT No.: PCT/IB2020/058737

§ 371 Date: Mar. 16, 2022

Related U.S. Application Data
(60) Provisional application No. 62/902,828, filed on Sep. 19, 2019.

(51) Int. Cl. A61K 31/575; A61K 31/46; A61K 31/4162; A61K 45/06

(52) U.S. Cl. CPC A61K 31/575; A61K 31/46; A61K 31/4162; A61K 45/06

(57) ABSTRACT

The invention provides, FXR agonists for the treatment of a condition mediated by Farnesoid X receptor (FXR), in particular liver disease or intestinal disease, in a subject in need thereof, to reduce drug-induced adverse side effects inpatients suffering from such diseases or conditions.

### FIELD OF THE INVENTION

The present invention relates to methods for treating, preventing, or ameliorating a condition mediated by farnesoid X receptors (FXRs), in particular liver diseases or intestinal diseases, comprising administering to a subject in need thereof a therapeutically effective amount of an FXR agonist, to reduce drug-induced adverse side effects in patients suffering from such diseases or conditions; as well as uses and compositions involving such regimens.

### BACKGROUND OF THE INVENTION

Nonalcoholic fatty liver disease (NAFLD) is the most common cause of chronic liver disease in the Western world. The main stages of NAFLD are 1—simple fatty liver (steatosis); 2—non-alcoholic steatohepatitis (NASH), a more serious form of NAFLD with fat accumulation accompanied by inflammation and cell injury; 3—fibrosis, where there is a persistent inflammation in the liver resulting in the generation of fibrous scar tissue around the liver cells and blood vessels; and 4-cirrhosis, wherein damage is permanent and can lead to liver failure and liver cancer (hepatocellular carcinoma).

FXR agonism has shown clinical benefits in subjects with cholestatic disorders (Nevens et al., J. Hepatol. 60 (1 SUPPL. 1): 347A-348A (2014)), bile acid malabsorption diarrhea (Walters et al., Aliment Pharmacol. Ther. 41(1):54-64 (2014)) and non-alcoholic steatohepatitis (NASH; Neuschwander-Tetri et al 2015). When tested in NASH patients, obeticholic acid (OCA), a bile acid mimetic, showed efficacy, in particular a significant improvement in NAS, i.e. strong impact on steatosis with additional effects on lobular inflammation and ballooning. However, long term administration of OCA was associated with pruritus and lipid abnormalities, i.e. increased low density lipoprotein (LDL) cholesterol; thereby raising safety concerns. (see Results from REGENERATE (NCT02548351), A Phase 3 International, Randomized, Placebo-Controlled Study Evaluating Obeticholic Acid Treatment for NASH, EASL 2019 April 10-14 Vienna).

The FXR agonist tropifexor (see Tully et al, J Med Chem 2017; 60:9960-9973) is currently tested in nonalcoholic steatohepatitis patients with fibrosis (see NCT02855164 study). The compound was disclosed for the first time in WO 2012/087519 (Example 1, compound 1-IB of the table on page 125) and it is known under the name LJN452.

Acetyl-CoA carboxylase (ACC) is an enzyme with two isoforms (ACC1 and ACC2) that is involved in de novo lipogenesis (the synthesis of endogenous fatty acids) and the regulation of beta-oxidation. Inhibitors of ACC therefore have the potential to prevent production of new lipids within the liver and stimulate their breakdown. In animal models of fatty liver, ACC inhibition reduces hepatic fat content, inflammation and fibrosis (scarring), all of which are important hallmarks of NASH progression. ACC inhibitor GS-0976, known under its international non-proprietary name firsocostat (2-[1-{(2R)-2-(2-methoxyphenyl)-2-[(oxan-4-yl)oxy]ethyl}-5-methyl-6-(1,3-oxazol-2-yl)-2,4-dioxo-1,4-dihydrothieno[2,3-d]pyrimidin-3(2H)-yl]-2-methylpropanoic acid disclosed for example in U.S. Pat. No. 8,969,557, WO2017151816), is being clinically tested in NASH subjects and has showed to improve steatosis and improvements in MRI-PDFF; however, in some patients ACC inhibition by GS-0976 has been associated with elevated plasma triglycerides (TG) (The Liver Meeting, abstract LB-9, 2017).

Currently there is no approved therapy for NASH. There remains a need to provide treatments for fibrotic/cirrhotic diseases or disorders, e.g. liver diseases or disorders that can address the different aspects of these complex conditions, in any patient in need of such treatment while demonstrating an acceptable safety and/or tolerability profile, and could be associated with more limited side effects which are drug-induced by treatment with ACC inhibitors.

### SUMMARY OF THE INVENTION

The present invention relates to methods for treating, preventing, or ameliorating a condition mediated by farnesoid X receptor (FXR), in particular liver diseases or intestinal diseases, comprising administering to a subject in need thereof a therapeutically effective amount of an FXR agonist; wherein administration of the FXR agonist reduces drug-induced adverse side effects in said subject.

In one aspect, the invention relates to methods for treating, preventing, or ameliorating a condition mediated by farnesoid X receptor (FXR), e.g. NASH, comprising administering to a subject in need thereof a therapeutically effective amount of an FXR agonist; wherein administration of the FXR agonist to said subject reduces drug-induced weight gain, e.g. weight gain induced by the administration of or treatment with an ACC inhibitor.

In another aspect, the invention provides a pharmaceutical combination comprising an FXR agonists and an ACC inhibitor.

In yet another aspect, the invention relates to methods for treating, preventing, or ameliorating a condition mediated by FXR, e.g. NASH, comprising administering to a subject in need thereof a therapeutically effective amount of an FXR agonist of formula

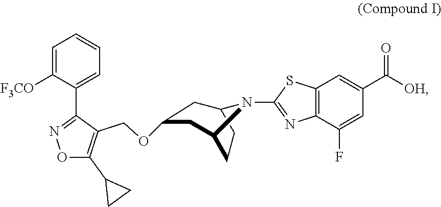

i.e. 2-[(1R,3r,5S)-3-({5-cyclopropyl-3-[2-(trifluoromethoxy)phenyl]-1,2-oxazol-4-yl}methoxy)-8-azabicyclo[3.2.1]octan-8-yl]-4-fluoro-1,3-benzothiazole-6-carboxylic acid) also known under its INN tropifexor, in free form or a pharmaceutically acceptable salt thereof or an amino acid conjugate thereof, wherein the administration of the FXR agonist to said subject reduces a drug-induced adverse side effect in said subject, such as drug-induced weight gain induced by the administration of, or the treatment with an ACC inhibitor. In one embodiment, the administration of the FXR agonist is occurring in the evening.

An advantage of the present invention is that the FXR agonist potentiates the therapeutic effect of ACC inhibitor, and reduces adverse side effects that are generally induced by the use of various ACC inhibitors. Side effects like weight gain are frequent side effects in patients who are treated with ACC inhibitors. As such, the patient can be administered a lower dose of a ACC inhibitors in combination with an FXR agonist and still achieve the same beneficial therapeutic effect normally associated with a higher dose of the same ACC inhibitors. Therefore, the treatment of patients with ACC inhibitors in combination with FXR agonists considerably alleviates ACC inhibitors associated side effects, like weight gain.

Thus, the treatment regimens according to the present invention offer the benefit of a high therapeutic efficacy while having low incidence of side effects, such as lipid abnormalities (e.g. increased LDL cholesterol), which have been observed while using ACC inhibitors treatment regimen. These treatment regimens further provide subjects with a convenient once daily dosing, thus supporting patient compliance.

### DETAILED DESCRIPTION OF THE INVENTION

Drug-induced weight gain is a serious side effect of drugs leading to noncompliance with therapy and to exacerbation of comorbid conditions related to obesity. It is a problematic side effect of therapy due to the known deleterious effect of weight gain on glucose control, increased blood pressure and worsening lipid profile. (Ness-Abramof et al., Drugs of Today 2005, 41(8): 547-555).

Hypertriglyceridemia mediated by ACC inhibition was reported in GS-0976 (firsocostat)—treated NASH patients or in healthy volunteers treated with MK-4704, another liver-targeted ACC inhibitor. Preclinical studies confirmed that the hypertriglyceridemia observed in human clinical trials were a direct consequence of ACC inhibition.

It has been found that administering an FXR agonist to a subject in need thereof reduces drug-induced adverse side effects in said subject, such as drug-induced weight gain, induced by the administration of, or the treatment with an ACC inhibitor. The co-administation of a farnesoid X receptor (FXR) agonist, e.g. tropifexor, reduces the adverse side effects of the ACC inhibitor; such co-administration is beneficial for therapeutic efficacy and for safety (such as reducing lipid abnormalities, weight gain and/or peripheral obesity). In contrast to ACC inhibition, treatment with FXR agonist LJP305 (compound described in Tully et al, J Med Chem 2017; 60:9960-9973) decreased body weight gain induced by the HF/NASH diet. The beneficial effects of LJP305 on reduction of body weight and peripheral fat were not a consequence of decreased food intake since even a transient increase in food intake was observed in the study.

Various (enumerated) embodiments of the present invention are described herein. It will be recognized that features specified in each embodiment may be combined with other specified features to provide further embodiments of the present disclosure.

### EMBODIMENTS

1. A method for treating a condition mediated by Farnesoid X receptor (FXR), in particular a liver disease or an intestinal disease, in a subject in need thereof, comprising administering once daily to said subject a therapeutically effective amount of an FXR agonist; wherein administration of the FXR agonist to said subject reduces drug-induced adverse side effects in said subject, such as drug-induced weight gain, e.g. induced by the administration of, or the treatment with an ACC inhibitor.

2. A method for the prevention of a condition mediated by Farnesoid X receptor (FXR), in particular a liver disease or an intestinal disease, in a subject in need thereof, comprising administering once daily to said subject a therapeutically effective amount of an FXR agonist;

wherein administration of the FXR agonist to said subject reduces drug-induced adverse side effects in said subject, such as drug-induced weight gain, e.g. induced by the administration of, or the treatment with an ACC inhibitor.

3. A method for the treatment, stabilization or lessening the severity or progression of a non-alcoholic fatty liver disease (NAFLD) in a subject in need thereof, comprising administering once daily to said subject a therapeutically effective amount of an FXR agonist; wherein administration of the FXR agonist to said subject reduces drug-induced adverse side effects in said subject, such as drug-induced weight gain, e.g. induced by the administration of, or the treatment with an ACC inhibitor.

4. A method for the treatment, stabilization or lessening the severity or progression of an intestinal disease in a subject in need thereof, comprising administering once daily to said subject a therapeutically effective amount of an FXR agonist; wherein administration of the FXR agonist to said subject reduces drug-induced adverse side effects in said subject, such as drug-induced weight gain, e.g. induced by the administration of, or the treatment with an ACC inhibitor.

5. A method for the treatment, stabilization or lessening the severity or progression of a non-alcoholic steatohepatitis (NASH) in a subject in need thereof, comprising administering once daily to said subject a therapeutically effective amount of an FXR agonist; wherein administration of the FXR agonist to said subject reduces drug-induced adverse side effects in said subject, such as drug-induced weight gain, e.g. induced by the administration of, or the treatment with an ACC inhibitor.

6. A method for slowing, arresting, or reducing the development of a chronic liver disease or disorder, e.g. NAFLD, NASH, liver fibrosis or PBC, in a subject in need thereof, comprising administering once daily to said subject a therapeutically effective amount of an FXR agonist;

wherein administration of the FXR agonist to said subject reduces drug-induced adverse side effects in said subject, such as drug-induced weight gain, e.g. induced by the administration of, or the treatment with an ACC inhibitor.

7. A method for reducing cirrhosis or fibrosis in a subject having a disease that is non-alcoholic fatty liver disease (NAFLD) or non-alcoholic steatohepatitis (NASH), comprising administering once daily to said subject a therapeutically effective amount of an FXR agonist; wherein administration of the FXR agonist to said subject reduces drug-induced adverse side effects in said subject, such as drug-induced weight gain, e.g. induced by the administration of, or the treatment with an ACC inhibitor.

8. The method according to any one of Embodiments 1 to 7, wherein said method further comprises lack of worsening of the subject's NAFLD as defined by Activity (NAS) score, lack of worsening of the subject's Steatosis, Activity and Fibrosis (SAF) Activity score, reduction of liver fat in said subject, improvement in subject's steatosis, improvement in subject's ballooning, NAFLD resolution, NAFLD resolution without worsening of fibrosis, reduction of fibrosis without NAFLD worsening, reduction of ALT levels in said subject, reduction of AST levels in said subject, reduction of HbA1c levels in said subject, lack of subject's progression to cirrhosis, inhibiting progression of non-alcoholic fatty liver disease (NAFLD) and/or non-alcoholic steatohepatitis (NASH), or any combination thereof.

9. The method according to any one of Embodiments 1 to 8, wherein the FXR agonist is selected from tropifexor, obeticholic acid, nidufexor, cilofexor, TERN-101, EDP-305, PXL007, AGN242266 and MET409.

10. The method according to Embodiment 9, wherein the FXR agonist is obeticholic acid.

11. The method according to Embodiment 10, wherein obeticholic acid is administered at a daily dose of about 5 mg, of about 10 mg, of about 15 mg, of about 20 mg, of about 25 mg, of about 30 mg, of about 40 mg, or of about 50 mg.

12. The method according to Embodiment 9, wherein the FXR agonist is tropifexor.

13. The method according to Embodiment 12, wherein tropifexor is administered at a daily dose of about 90 μg to about 250 μg, e.g. of about 140 μg to about 200 μg.

14. The method according to Embodiment 12, wherein tropifexor is administered at a dose of about 90 μg/day, of about 140 μg/day, of about 150 μg/day, of about 160 μg/day, of about 170 μg/day, of about 180 μg/day, of about 190 μg/day, of about 200 μg/day, of about 210 μg/day, of about 220 μg/day, of about 230 μg/day, of about 240 μg/day or of about 250 μg/day.

15. The method according to Embodiment 12 wherein tropifexor is administered at a daily dose of about 140 μg.

16. The method according to Embodiment 9, wherein the FXR agonist is cilofexor.

17. The method according to Embodiment 16, wherein cilofexor is administered at a daily dose of about 5 mg, of about 10 mg, of about 15 mg, of about 20 mg, of about 25 mg, of about 30 mg, of about 40 mg or of about 50 mg.

18. The method according to Embodiment 16 wherein cilofexor is administered at a daily dose of about 30 mg, once daily.

19. The method according to any one of Embodiments 1 to 18, wherein the FXR agonist is administered in the evening.

20. The method according to Embodiment 19, wherein said evening administration reduces the risk of side effects, e.g. pruritus, associated with administration of the FXR agonist.

21. The method according to Embodiment 19, wherein said evening administration reduces the risk of side effects, e.g. lipid abnormality, associated with administration of the FXR agonist.

22. The method according to any one of Embodiments 1 to 21, wherein said administration comprises resolution of steatohepatitis, e.g. NASH.

23. The method according to any one of Embodiments 1 to 21, wherein said administration comprises improvement in liver fibrosis.

24. The method according to any one of Embodiments 1 to 21, wherein said administration comprises resolution of steatohepatitis, e.g. NASH, and improvement in liver fibrosis.

25. The method according to any one of Embodiments 1 to 24, wherein the ACC inhibitor is selected from firsocostat, MK-4074 and PF-05221304.

26. The method according to Embodiment 25, wherein the ACC inhibitor is firsocostat.

27. The method according to Embodiment 26, wherein firsocostat is administered at a dose of about 5 mg, of about 10 mg, of about 15 mg, of about 20 mg, of about 25 mg, of about 30 mg, of about 40 mg or of about 50 mg.

28. The method according to Embodiment 26, wherein firsocostat is administered at a daily dose of about 20 mg, once daily.

29. The method according to any one of Embodiments 1 to 28 comprising administering to a subject in need thereof a combination of an ACC inhibitor and an FXR agonist.

30. A method, or a pharmaceutical composition comprising a combination of an ACC inhibitor and an FXR agonist, according to any one of above listed Embodiments, for treating or preventing non-alcoholic steatohepatitis (NASH), and wherein NASH is mild to moderate with fibrosis level F2-F3.

31. A method, or a pharmaceutical composition comprising a combination of an ACC inhibitor and an FXR agonist, according to any one of above listed Embodiments, wherein NASH is confirmed based on liver biopsy (also called biopsy-proven NASH) and NASH is mild to moderate with fibrosis level F2-F3.

32. A method, or a pharmaceutical composition comprising a combination of an ACC inhibitor and an FXR agonist according to any one of the above listed Embodiments, wherein presence of NASH has been demonstrated by:

i) Histologic evidence of NASH based on liver biopsy obtained 2 years or less before treatment with an FXR agonist according to any one of the above Embodiments, with a diagnosis consistent with NASH, fibrosis level F1, F2, F3 or F4, no diagnosis of alternative chronic liver diseases, or

ii) Phenotypic diagnosis of NASH, or

iii) Noninvasive, disease-specific biomarkers.

33. A method, or a pharmaceutical composition comprising a combination of an ACC inhibitor and an FXR agonist according to any one of the above listed Embodiments, for reducing adverse side effects, e.g. weight gain and peripheral obesity induced by an ACC inhibitor, by co-administering an farnesoid X receptor (FXR) agonist, e.g. tropifexor, to reduce the adverse side effects of the ACC inhibitor, e.g. firsocostat.

34. A combination comprising a therapeutically effective amount of an FXR agonist and an ACC inhibitor.

35. A pharmaceutical combination comprising a therapeutically effective amount of an FXR agonist and an ACC inhibitor, for simultaneous, sequential or separate administration.

36. A method, combination or pharmaceutical combination according to any one of the above listed Embodiments, wherein the FXR agonist is tropifexor. In particular embodiments, tropifexor is administered at a dose (e.g. daily dose) of about 90 μg to about 250 μg, e.g. of about 140 μg to about 200 μg.

37. A method, combination or pharmaceutical combination according to any one of the above listed Embodiments, wherein the FXR agonist is obeticholic acid. In particular embodiments, obeticholic acid is administered at a daily dose of about 5 mg, of about 10 mg, of about 15 mg, of about 20 mg, of about 25 mg, of about 30 mg, of about 40 mg, or of about 50 mg.

38. A method, combination or pharmaceutical combination according to any one of the above listed Embodiments, wherein the FXR agonist is cilofexor. In some embodiments, cilofexor is administered at a daily dose of about 5 mg, of about 10 mg, of about 15 mg, of about 20 mg, of about 25 mg, of about 30 mg, of about 40 mg or of about 50 mg. In particular embodiments, cilofexor is administered at a daily dose of about 30 mg, once daily.

39. A method, combination or pharmaceutical combination according to any one of the above listed Embodiments, wherein the FXR agonist is firsocostat. In some embodiments, firsocostat is administered at a daily dose of about 5 mg, of about 10 mg, of about 15 mg, of about 20 mg, of about 25 mg, of about 30 mg, of about 40 mg or of about 50 mg. In particular embodiments, firsocostat is administered at a daily dose of about 20 mg, once daily.

In some aspects, the FXR agonists as defined herein, are provided for the treatment of a disease or disorder mediated by FXR, e.g. a liver disease or disorder, e.g. a chronic liver disease or disorder, e.g. a disease or disorder selected from the group consisting of cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis, e.g. NAFLD, NASH, hepatic fibrosis, hepatosteatis or PBC.

In yet another aspect, a pharmaceutical unit dosage form composition comprising about 90 μg, about 140 μg, about 150 μg, about 160 μg, about 170 μg, about 180 μg, about 190 μg, about 200 μg, about 210 μg, about 220 μg, about 230 μg, about 240 μg or about 250 μg of tropifexor suitable for oral administration once daily, in the evening, or shortly before or at bedtime. Such unit dosage form compositions may be in a form selected from a liquid, a tablet, a capsule. Also these unit dosage form compositions are for use in treating a chronic liver disease, e.g. non-alcoholic fatty liver disease (NAFLD), non-alcoholic steatohepatitis (NASH), drug-induced bile duct injury, gallstones, liver cirrhosis, alcohol-induced cirrhosis, cystic fibrosis, bile duct obstruction, cholelithiasis, liver fibrosis, e.g. for use in treating non-alcoholic steatohepatitis (NASH), e.g. for use in treating phenotypic non-alcoholic steatohepatitis (NASH).

In yet another aspect, the FXR agonists as defined herein are provided for preventing or delaying progression of a chronic liver disease or disorder to a more advanced stage or a more serious condition thereof, e.g. for preventing or delaying progression of a chronic liver disease or disorder selected from the group consisting of NAFLD, NASH, hepatic fibrosis and PBC.

### Definitions

For purposes of interpreting this specification, the following definitions will apply and whenever appropriate, terms used in the singular will also include the plural and vice versa.

As used herein, the term “about” in relation to a numerical value×means +/−10%, unless the context dictates otherwise.

As used herein, a “ACC inhibitor”/“ACC inhibitors” refer to any agent that is capable of inhibiting Acetyl-CoA carboxylase, capable of inhibiting at least one of its two isoforms (ACC1 and ACC2). Several systemic ACC inhibitors have been disclosed in recent years (Griffith et al. J Med Chem, 2014, 57 (24), 10512-26).

The ACC inhibitor is preferably selected from:
Firsocostat (also known under the name GS-0976 or ND-630; see Lawitz et al. Clin Gastroenterol Hepatol, 2018, 16 (12), 1983-1991)

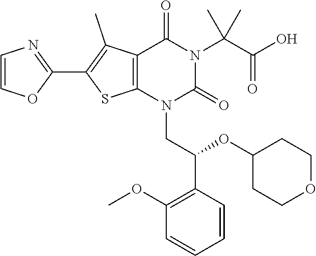 [SMILES: COc1ccccc1[C@H](Cn1c(=O)n(C(C)(C)C(=O)O)c(=O)c2c(C)c(-c3ncco3)sc21)OC1CCOCC1]

MK-4074 (Merck; see Kim et al. Cell Metab, 2017, 26 (2), 394-406)

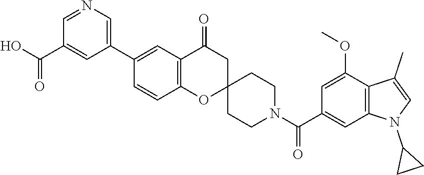 [SMILES: COc1cc(C(=O)N2CCC3(CC2)CC(=O)c2cc(-c4cncc(C(=O)O)c4)ccc2O3)cc2c1c(C)cn2C1CC1]

Pfizer (see Freeman-cook et al J Med Chem, 2012, 55 (2), 935-4);

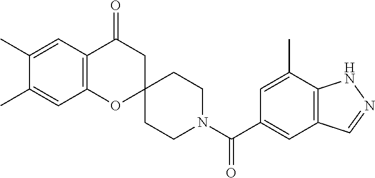 [SMILES: Cc1cc2c(cc1C)C(=O)CC1(CCN(C(=O)c3cc(C)c4[nH]ncc4c3)CC1)O2]

Pfizer (2012; see Griffith et al. J Med Chem, 2014, 57 (24), 10512-26)

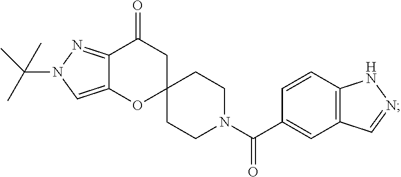 [SMILES: CC(C)(C)n1cc2c(n1)C(=O)CC1(CCN(C(=O)c3ccc4[nH]ncc4c3)CC1)O2]

Pfizer (2014; see Griffith et al. J Med Chem, 2014, 57 (24), 10512-26)

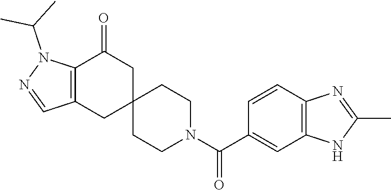 [SMILES: Cc1nc2ccc(C(=O)N3CCC4(CC3)CC(=O)c3c(cnn3C(C)C)C4)cc2[nH]1]

and
PF-05221304 (Pfizer)

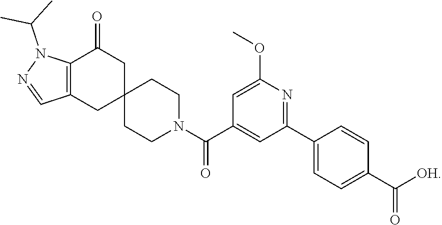 [SMILES: COc1cc(C(=O)N2CCC3(CC2)CC(=O)c2c(cnn2C(C)C)C3)cc(-c2ccc(C(=O)O)cc2)n1]

As used herein, a “FXR agonist”/“FXR agonists” refer to any agent that is capable of binding and activating farnesoid X receptor (FXR) which may be referred to as bile acid receptor (BAR) or NR1H4 (nuclear receptor subfamily 1, group H, member 4) receptor. FXR agonist may act as agonists or partial agonists of FXR. The agent may be e.g. a small molecule, an antibody or a protein, preferably a small molecule. The activity of an FXR agonist may be measured by several different methods, e.g. in an in vitro assay using the fluorescence resonance energy transfer (FRET) cell free assay as described in Pellicciari, et al. J. Med. Chem., 2002 vol. 15, No. 45:3569-72.

The FXR agonist as used herein refers, for example, to compounds disclosed in: WO2016/096116, WO2016/127924, WO2017/218337, WO2018/024224, WO2018/075207, WO2018/133730, WO2018/190643, WO2018/214959, WO2016/096115, WO2017/118294, WO2017/218397, WO2018/059314, WO2018/085148, WO2019/007418, CN109053751, CN104513213, WO2017/128896, WO2017/189652, WO2017/189663, WO2017/189651, WO2017/201150, WO2017/201152, WO2017/201155, WO2018/067704, WO2018/081285, WO2018/039384, WO2015/138986, WO2017/078928, WO2016/081918, WO2016/103037, WO2017/143134.

The FXR agonist is preferably selected from: tropifexor, nidufexor, obeticholic acid (6α-ethyl-chenodeoxycholic acid), cilofexor (GS-9674, Px-102), MET409, AGN242266,
TERN-101 (LY2562175):

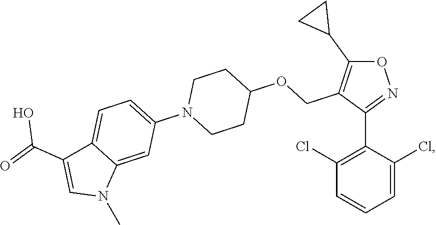 [SMILES: Cn1cc(C(=O)O)c2ccc(N3CCC(OCc4c(-c5c(Cl)cccc5Cl)noc4C4CC4)CC3)cc21]

PXL007 (EYP001)

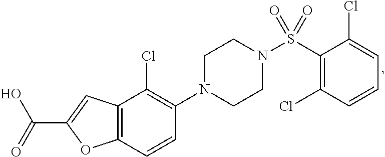 [SMILES: O=C(O)c1cc2c(Cl)c(N3CCN(S(=O)(=O)c4c(Cl)cccc4Cl)CC3)ccc2o1]

and
EDP-305:

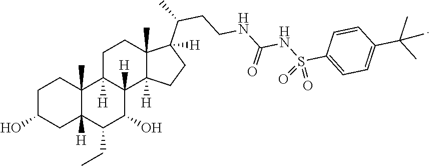

As used herein, the terms “salt” or “salts” refer to an acid addition or base addition salt of a compound of the invention. “Salts” include in particular “pharmaceutical acceptable salts”, and both can be used interchangeably herein.

As used herein, the term “pharmaceutically acceptable” means a nontoxic material that does not substantially interfere with the effectiveness of the biological activity of the active ingredient(s).

As used herein the term “prodrug” refers to a compound that is converted in vivo to the compounds of the present invention. A prodrug is active or inactive. It is modified chemically through in vivo physiological action, such as hydrolysis, metabolism and the like, into a compound of this invention following administration of the prodrug to a subject. The suitability and techniques involved in making and using pro-drugs are well known by those skilled in the art. Suitable prodrugs are often pharmaceutically acceptable ester derivatives.

As used herein, the terms “subject” or “subjects” refer to a mammalian organism, preferably a human being, who is diseased with the condition (i.e. disease or disorder) of interest and who would benefit from the treatment, e.g. a patient.

As used herein, a subject is “in need of” a treatment if such subject would benefit biologically, medically or in quality of life from such treatment.

As used herein, the term “treat”, “treating” or “treatment” of any disease or disorder refers in one embodiment to ameliorating the disease or disorder (i.e. slowing or arresting or reducing the development of the disease or at least one of the clinical symptoms or pathological features thereof). In another embodiment “treat”, “treating” or “treatment” refers to alleviating or ameliorating at least one physical parameter or pathological features of the disease, e.g. including those, which may not be discernible by the subject. In yet another embodiment, “treat”, “treating” or “treatment” refers to modulating the disease or disorder, either physically, (e.g. stabilization of at least one discernible or non-discernible symptom), physiologically (e.g. stabilization of a physical parameter) or both. In yet another embodiment, “treat”, “treating” or “treatment” refers to preventing or delaying the onset or development or progression of the disease or disorder, or of at least one symptoms or pathological features associated thereof. In yet another embodiment, “treat”, “treating” or “treatment” refers to preventing or delaying progression of the disease to a more advanced stage or a more serious condition, such as e.g. liver cirrhosis; or to preventing or delaying a need for liver transplantation.

As used herein, the term “nonalcoholic fatty liver disease” (NAFLD) may refer to nonalcoholic fatty liver (NAFL), noncirrhotic NASH, and NASH with cirrhosis.

For example, “treating” NASH may refer to ameliorating, alleviating or modulating at least one of the symptoms or pathological features associated with NASH; e.g. hepatosteatosis, hepatocellular ballooning, hepatic inflammation and fibrosis; e.g. may refer to slowing progression, reducing or stopping at least one of the symptoms or pathological features associated with NASH, e.g. hepatosteatosis, hepatocellular ballooning, hepatic inflammation and fibrosis. It may also refer to preventing or delaying liver cirrhosis or a need for liver transplantation, e.g. slow the progress of, halt, or reverse disease progression and improve clinical outcomes (i.e., prevent progression to cirrhosis and 283 cirrhosis complications, reduce the need for liver transplantation, and improve survival)

Also “treating” NASH may refer to slow the progress of, halt, or reverse disease progression and improve clinical outcomes i.e., prevent progression to cirrhosis and Resolution of steatohepatitis and no worsening of liver fibrosis on NASH clinical research network (CRN) histological score.

The treatment of NASH includes:
“Resolution of steatohepatitis” is defined as absence of fatty liver disease or isolated or simple steatosis without steatohepatitis and a NAS score of 0-1 for inflammation, 0 for ballooning, and any value for steatosis; cirrhosis complications, reduction in the need for liver transplantation, and improved survival;
Or Improvement in liver fibrosis greater than or equal to one stage (NASH CRN histological score) and no worsening of steatohepatitis (e.g. defined as no increase in NAS for ballooning, inflammation, or steatosis);
Or Both resolution of steatohepatitis and improvement in fibrosis (as defined above). “Treating” or “treatment” of NAFLD or NASH in a human includes one or more of:
a) Reducing the risk of developing NAFLD or NASH, i.e., causing clinical symptoms of NAFLD or NASH not to develop in a subject who may be predisposed to NAFLD or NASH
b) Inhibiting NAFLD or NASH, i.e., arresting or reducing the development of NALFD or NASH or its clinical symptoms; and
c) Relieving NAFLD or NASH, i.e., causing regression, reversal, or amelioration of the NAFLD or NASH or reducing number, frequency, duration or severity of its clinical symptoms.

As used herein, the term “prevent”, “preventing” or “prevention” in connection to a disease or disorder refers to the prophylactic treatment of a subject who is at risk of developing a condition (e.g., specific disease or disorder or clinical symptom thereof) resulting in a decrease in the probability that the subject will develop the condition.

As used herein, the term “therapeutically effective amount” refers to an amount of the compound, which is sufficient to achieve the stated effect. Accordingly, a therapeutically effective amount used for the treatment or prevention of a liver disease or disorder as hereinabove defined is an amount sufficient for the treatment or prevention of such a disease or disorder.

By “therapeutic regimen” is meant the pattern of treatment of an illness, e.g., the pattern of dosing used during the treatment of the disease or disorder.

As used herein, the term “liver disease or disorder” encompasses one, a plurality, or all of non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis and liver fibrosis.

As used herein, the term NAFLD may encompass the different stages of the disease: hepatosteatosis, NASH, fibrosis and cirrhosis.

As used herein, the term NASH may encompass steatosis, hepatocellular ballooning and lobular inflammation.

As herein defined, “combination” refers to either a fixed combination in one unit dosage form (e.g., capsule, tablet, or sachet), free (i.e. non-fixed) combination, or a kit of parts for the combined administration where an FXR agonist, such as tropifexor, and the one or more additional therapeutic agents may be administered independently at the same time or separately within time intervals, especially where these time intervals allow that the combination partners show a cooperative, e.g. synergistic effect.

The terms “co-administration” or “combined administration” or the like, as utilized herein, are meant to encompass administration of an additional therapeutic agent to a single subject in need thereof (e.g. a subject), and the additional therapeutic agent are intended to include treatment regimens in which the FXR agonist and additional therapeutic agent are not necessarily administered by the same route of administration and/or at the same time. Each of the components of the combination of the present invention may be administered simultaneously or sequentially and in any order. Co-administration comprises simultaneous, sequential, overlapping, interval, continuous administrations and any combination thereof.

The term “pharmaceutical combination” as used herein means a pharmaceutical composition that results from the combining (e.g. mixing) of more than one active ingredient and includes both fixed and free combinations of the active ingredients.

The term “fixed combination” means that the active ingredients are administered to a subject simultaneously in the form of a single entity or dosage.

The term “free combination” means that the active ingredients as herein defined are administered to a subject as separate entities either simultaneously, concurrently or sequentially with no specific time limits, and in any order, wherein such administration provides therapeutically effective levels of the compounds in the subject's body.

By “simultaneous administration”, it is meant that the active ingredients as herein defined, are administered on the same day. The active ingredients can be administered at the same time (for fixed or free combinations), or one at a time (for free combinations).

According to the invention, “sequential administration”, may mean that during a period of two or more days of continuous co-administration only one of the active ingredients as herein defined, is administered on any given day.

By “overlapping administration”, it is meant that during a period of two or more days of continuous co-administration, there is at least one day of simultaneous administration and at least one day when only one of active ingredients as herein defined, is administered.

By “continuous administration”, it is meant a period of co-administration without any void day. The continuous administration may be simultaneous, sequential, or overlapping, as described above.

As used herein, the term “qd” means a once daily administration.

The term “dose” refers to a specified amount of a drug administered at one time. As used herein, the dose is the amount of the drug that elicits a therapeutic effect. The dose would, for example, be declared on a product package or in a product information leaflet. For example, for tropifexor, the term “dose” when used in relation to tropifexor is the amount of tropifexor in free form. Since tropifexor can be present in the form of a salt or of an amino acid conjugate, the amount of the respective salt former (e.g. the respective acid) or of the amino acid, is added accordingly.

The term “weight gain”, as used herein, refers to an increased body mass that may be drug-induced. As such, the weight gain in a person may be the result of administering a specific drug to that person. In order to determine whether a person is overweight or obese, a body mass index (BMI) is used. The BMI is a measure used to evaluate body weight relative to height. Hence, the BMI can be used to find out whether a person is underweight, normal weight, overweight, or obese. See Body Mass Index Table of Clinical Guidelines on the Identification, Evaluation, and Treatment of Overweight and Obesity in Adults: The Evidence Report (Insulin Resistance and Pre-Diabetes, NIH Publication No. 04-4893 (May 2004).

### Modes of Administration

The pharmaceutical composition of the invention can be formulated to be compatible with its intended route of administration (e.g. oral compositions generally include an inert diluent or an edible carrier). Other non-limiting examples of routes of administration include parenteral (e.g. intravenous), intradermal, subcutaneous, oral (e.g. inhalation), transdermal (topical), transmucosal, and rectal administration. The pharmaceutical compositions compatible with each intended route are well known in the art.

### Timing of the Administration

The FXR agonist of the invention, as herein defined in above listed embodiments, is administered in the evening.

In one embodiment, the term “administration in the evening” is generally defined as administration any time from about 6 pm to about 12 pm, e.g. from about 8 pm to about 11 pm, preferably around 9 pm. Administration in the evening may be before the evening meal, with the evening meal or after the evening meal.

In one embodiment, the term “administration in the evening” refers to administration shortly before or at bedtime. In one embodiment, the term “administration in the evening” refers to administration shortly before bedtime. In one embodiment, the term “administration in the evening” refers to administration at bedtime. Unless otherwise specified herein, the term “bedtime” has the normal meaning of a time when a person retires for the primary sleep period during a twenty-four hour period of time. The administration shortly before bedtime means that the FXR agonist as herein defined, is administered within about 1-2 hours prior to a person's normal rest or sleep (typically 4 to 10-hours) period.

### Diseases

As hereinabove defined, the fibrotic or cirrhotic disease or disorder can be a liver disease or disorder, e.g. as defined herein, or renal fibrosis.

As hereinabove defined, the liver diseases or disorders can be cholestasis, intrahepatic cholestasis, estrogen-induced cholestasis, drug-induced cholestasis, cholestasis of pregnancy, parenteral nutrition-associated cholestasis, primary biliary cirrhosis (PBC), primary sclerosing cholangitis (PSC), progressive familiar cholestasis (PFIC), non-alcoholic fatty liver disease (NAFLD), non-alcoholic steatohepatitis (NASH), drug-induced bile duct injury, gallstones, liver cirrhosis, alcohol-induced cirrhosis, cystic fibrosis-associated liver disease (CFLD), bile duct obstruction, cholelithiasis, liver fibrosis, renal fibrosis, dyslipidemia, atherosclerosis, diabetes, diabetic nephropathy, colitis, newborn jaundice, prevention of kernicterus, veno-occlusive disease, portal hypertension, metabolic syndrome, hypercholesterolemia, intestinal bacterial overgrowth, erectile dysfunction, progressive fibrosis of the liver caused by any of the diseases above or by infectious hepatitis. The liver diseases or disorders can also refer to liver transplantation.

As hereinabove defined, the intestinal disease can be idiopathic inflammatory bowel disease, e.g. Crohn's disease or ulcerative colitis.

In one embodiment of the invention, the pharmaceutical compositions (as herein defined) are for the treatment or prevention of a fibrotic disease or disorder, e.g. a liver disease or disorder, e.g. a chronic liver disease, e.g. a liver disease or disorder selected from the group consisting of PBC, NAFLD, NASH, drug-induced bile duct injury, gallstones, liver cirrhosis, alcohol-induced cirrhosis, cystic fibrosis-associated liver disease (CFLD), bile duct obstruction, cholelithiasis, liver fibrosis. In one embodiment of the invention, the pharmaceutical combination (as herein defined) is for the treatment or prevention of fibrosis, e.g. renal fibrosis or liver fibrosis.

According to one embodiment of the invention, the liver diseases or disorders refer to NAFLD, e.g. any stages of NAFLD, e.g. any of steatosis, NASH, fibrosis and cirrhosis.

In one embodiment of the invention, there is provided a combination comprising an FXR agonist and an ACC inhibitor, as herein defined in above listed embodiments for the improvement of liver fibrosis without worsening of steatohepatitis.

In another embodiment of the invention, there is provided an FXR agonist of the invention, as herein defined in above listed embodiments, for obtaining a complete resolution of steatohepatitis without worsening, e.g. improving, of liver fibrosis.

In another embodiment of the invention, there is provided a combination comprising an FXR agonist and an ACC inhibitor, as herein defined in above listed embodiments, for preventing or treating steatohepatitis and liver fibrosis.

In yet another embodiment of the invention, there is provided a combination comprising an FXR agonist and an ACC inhibitor, as herein defined in above listed embodiments for reducing at least one of the features of the NAS score, i.e. one of hepatosteatosis, hepatic inflammation and hepatocellular ballooning; e.g. at least two features of the NAS score, e.g. hepatosteatosis and hepatic inflammation, or hepatosteatosis and hepatocellular ballooning, or hepatocellular ballooning and hepatic inflammation.

In a further embodiment of the invention, there is provided a combination comprising an FXR agonist and an ACC inhibitor, as herein defined in above listed embodiments, for reducing at least one or two features of the NAS score and liver fibrosis, e.g. for reducing hepatic inflammation and liver fibrosis, or hepatosteatosis and liver fibrosis or hepatocellular ballooning and liver fibrosis.

In yet a further embodiment of the invention there is provided a combination comprising an FXR agonist and an ACC inhibitor, as herein defined, for treating or preventing, stage 3 fibrosis to stage 1 fibrosis, e.g. stage 3 and/or stage 2 and/or stage 1 fibrosis.

In yet a further embodiment of the invention there is provided a combination comprising an FXR agonist and an ACC inhibitor, as herein defined, in above listed embodiments for treating or preventing an intestinal disease, e.g. idiopathic inflammatory bowel disease, e.g. Crohn's disease and ulcerative colitis.

The efficacy of the FXR agonist to reduce the drug-induced weight-gain, e.g induced by the administration of, or the treatment with ACC inhibitor, can be determined by monitoring subject's body weight and by calculating subject's BMI before and after the treatment with FXR agonist and ACC inhibitor. The subject may experience less than anticipated or no weight gain.

### Subjects

According to the invention, the subjects receiving the FXR agonist of the invention or the combination comprising an FXR agonist and an ACC inhibitor, as herein defined, can be affected or at risk of a fibrotic disease or disorder, e.g. a liver disease or disorder, e.g. as hereinabove defined.

According to the invention, the subjects receiving the FXR agonist of the invention or the combination comprising an FXR agonist and an ACC inhibitor, can have a drug-induced side effect, e.g. induced by the treatment with an ACC inhibitor, such as weight gain, wherein the weight gain is drug-induced by the treatment with an ACC inhibitor.

In some embodiments of the invention, the subject is obese or overweight.

In other embodiments of the invention, the subject may be a diabetic subject, e.g. may have type 2 diabetes. The subject may have high blood pressure and/or high blood cholesterol level.

### Dosing Regimens

Depending on the compound used, the targeted disease or disorder and the stage of such disease or disorder, the dosing regimen, i.e. administered doses and/or frequency of each component of the pharmaceutical combination may vary. The dosing frequency will depend on;

inter alia, the phase of the treatment regimen.

According to the invention, tropifexor (as hereinabove defined), is administered at a dose of about 90 μg to about 250 μg, e.g. about 140 μg to about 200 μg, e.g. about 140 μg. Such doses may be for oral administration. Preferably, tropifexor (as hereinabove defined), is administered at a dose of about 90 μg, or of about 140 μg.

In some aspects, tropifexor (as hereinabove defined), is administered at a dose of about 90 μg, about 100 μg, about 110 μg, about 120 μg, about 140 μg, or about 200 μg. Such doses are particularly adapted for oral administration of tropifexor.

In some embodiments, tropifexor is administered at a dose of about 120 μg delivered orally, of about 140 μg delivered orally, or of about 200 μg delivered orally.

In some embodiments, tropifexor is administered at a daily dose of about 90 μg.

In some embodiments, tropifexor is administered at a daily dose of about 120 μg.

In some embodiments, tropifexor is administered at a daily dose of about 140 μg.

In some embodiments, tropifexor is administered at a daily dose of about 200 μg.

In some aspects, obeticholic acid is administered at a daily dose of about 5 mg, of about 10 mg, of about 15 mg, of about 20 mg, of about 25 mg, of about 30 mg, of about 40 mg or of about 50 mg. In some embodiments, obeticholic acid is administered at a daily dose of about 25 mg.

### Dosing of ACC Inhibitor

In some embodiments, the total daily dose of the ACC inhibitor is selected from about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1 150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, about 2400 mg, about 2450 mg, about 2500 mg, about 2550 mg, about 2600 mg, about 2650 mg, about 2700 mg, about 2750 mg, about 2800 mg, about 2850 mg, about 2900 mg, about 2950 mg, or about 3000 mg.

In some embodiments, the total daily dose of the ACC inhibitor is between about 10 mg to about 3000 mg, between about 10 mg to about 2000 mg, between about 10 mg to about 1000 mg, between about 20 mg to about 1000 mg, between about 30 mg to about 1000 mg, between about 30 mg to about 750 mg, between about 30 mg to about 500 mg, between about 30 mg to about 250 mg, between about 30 mg to about 100 mg, between about 50 mg to about 500 mg, and between about 50 mg to about 100 mg.

### Unit Dosage Forms of ACC Inhibitor

The ACC inhibitor is preferably formulated in unit dosage form for ease of administration and uniformity of dosage. It will be understood, however, that the total daily usage of the ACC inhibitor and compositions thereof, will be decided by the attending physician within the scope of sound medical judgment. The specific effective dose level for any particular patient or organism will depend upon a variety of factors including the disorder being treated and the severity of the disorder; the specific composition employed; the age, body weight, general health, sex and diet of the patient; the time of administration, route of administration, and rate of excretion of a given ACC inhibitor; the duration of the treatment; drugs used in combination or coincidental with the ACC inhibitor, and like factors well known in the medical arts. A person of ordinary skill will appreciate that the unit dosage forms described herein refer to an amount of an ACC inhibitor, which may be provided as the free acid or free base or as a pharmaceutically acceptable salt thereof.

In some embodiment, the ACC inhibitor is administered in unit dosage formulations that comprise between about 5 mg to about 1000 mg of ACC inhibitor. In certain embodiments, a unit dosage formulation of the present invention provides about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 550 mg, about 600 mg, about 650 mg, about 700 mg, about 750 mg, about 800 mg, about 850 mg, about 900 mg, about 950 mg, or about 1000 mg of ACC inhibitor.

In some embodiments, the ACC inhibitor is administered in unit dosage formulations that comprise about 5 mg, about 30 mg, or about 150 mg of ACC inhibitor. In certain embodiments, a capsule formulation of the present invention provides about 25 mg, about 50 mg, about 75 mg, about 100 mg, about 125 mg, or about 150 mg of ACC inhibitor. In certain embodiments, an ACC inhibitor is administered at dosage levels of about 0.01 mg/kg to about 50 mg/kg and preferably from about 1 mg/kg to about 25 mg/kg, of subject body weight per day, one or more times a day, to obtain the desired therapeutic effect.

### EXAMPLES

### Example 1. Animal Studies

Studies were performed in compliance with Swiss guidelines for animal experimentation. Adult male C57BLJ6J mice were housed with ad libitum access to water and food. Mice were fed a HF/NASH diet (40 kcal % fat, 2% cholesterol, 40 kcal % carbohydrate, Research Diets, D09100301 or SSniff Special Diets, supplemented with a fructose-sucrose solution (42 g/L, 55% fructose and 45% sucrose by weight) in drinking water).

Total body fat and lean mass were analyzed by a nuclear magnetic resonance (NMR) and liver fat was measured by magnetic resonance imaging (MRI). Mice on HF/NASH diet did not gain weight and total fat mass on a uniform basis: mice gaining less than 10 g body weight and 7 g total body fat at week 7 HF/NASH diet (HF/NASH slow progressors, ˜15-20% of all animals) were excluded from intervention studies but analyzed for progression to NASH and HCC. Dietary intervention studies: mice fed a HF/NASH diet for 8 weeks were switched to normal diet (ND and water) for 12 weeks.

Drug treatment studies: mice exposed to HF/NASH diet for 20 weeks were treatment with LJP305 (1 mg/kg), GS-0976 (10 mg/kg) or vehicle (0.5% methylcellulose, 0.5% Tween 80 in water) p.o. once daily for 12 weeks while remaining on HF/NASH diet. At the end of the studies, blood and tissue samples were collected for analyses.

Liver samples were analyzed for collagen (Quickzyme), lipids and triglycerides (Cayman). Relative gene expression was calculated by normalizing Ct values of genes of interest against 18s (liver) or Idh3b (fat, ileum). Serum TIMP-1 (R&D Systems Inc.), PIII-NP (N-terminal procollagen III propeptide) (Cusabio Biotech Co.), α-fetoprotein (R&D Systems Inc.), leptin, and adiponectin (Meso Scale Discovery) were measured.

Paraffin-embedded tissue was stained with hematoxylin/eosin (H&E) and picrosirius red. IBA1 (ionized calcium-binding adapter molecule 1)-positive crown-like structures (Abcam, ab178846), Ly6b (BioRad, MCA771), UCP-1 (Abcam, ab109839) or ZO-1 (Invitrogen, 14-9776-80) were stained (Ventana Discovery XT) followed by digital (Aperio ScanScope XT, Leica Biosystems) and automated image analysis (HALO, Indica Labs).

C57BL/6J mice on a high fat diet, combined with ad libitum consumption of fructose-sucrose solution, developed several of the liver histological features seen in human disease, including steatosis, inflammation, and fibrosis, accompanied by elevated fibrosis biomarkers and liver injury enzymes. Obesity and metabolic impairments were associated with increased intestinal permeability and progression to adenoma and hepatocellular carcinoma.

Therapeutic treatment with ACC inhibitor GS-0976 or FXR agonist LJP305 resolved established NASH with fibrosis but had differential effects on liver transcriptome and peripheral obesity: ACC inhibition resulted in elevated blood triglycerides and peripheral obesity, FXR activation prevented peripheral obesity in NASH mice.

Surprisingly, chronic treatment with ACC inhibitor GS-0976 resulted in a significant increase in body weight compare to vehicle-treated animals despite similar food intake. At the end of the 12-week treatment, the mean body weight of GS-0976-treated animals was 7% above vehicle-treated mice. Consistently, GS-0976 treatment significantly increased total fat mass (27% vs vehicle), visceral and subcutaneous fat (30% vs vehicle), and substantially elevated circulating leptin levels (2-fold vs vehicle). In contrast, treatment with the FXR agonist LJP305 improved all measured metabolic readouts, as shown by a sustained decrease in body weight gain (7% vs vehicle at week 32 of HF/NASH) and a reduction in visceral and subcutaneous fat (23% and 26% vs vehicle, respectively). The FXR agonist LJP305 was more effective than ACC inhibiton in these experimental conditions with regards to improvement of macrovesicular steatosis and reducing peripheral obesity.

Results obtained in this study with the ACC inhibitor, GS-0976, mirror related clinical phase 2 NASH studies, where administration of GS-0976 for 12 weeks led to reductions in liver fat, serum TIMP-1, and liver injury. GS-0976 induced multiple genes known to play roles in lipid metabolism, among them genes catalyzing the synthesis of monounsaturated fatty acids, diacylglycerols, and triglycerides. This transcriptional shift in lipid metabolism could explain the histopathological findings and provide additional biological context as to why ACC inhibition is associated with fewer lipid microdroplets but stable levels of lipid macrodroplets.

Given that ACC inhibition and FXR activation exert both synergistic and complementary effects on NASH livers and obesity, this study provides a rationale for ACC/FXR combination therapy in NASH.

### Example 2. Role of Tropifexor in the Reductions of Hepatic Fat and Serum Alanine Aminotransferase in Patients with Fibrotic NASH after 12 Weeks of Therapy (FLIGHT-FXR Part C Interim Results)

FLIGHT-FXR (NCT02855164) is a phase 2 randomized, double blind, placebo-controlled, 3-part, adaptive-design study to assess the safety, tolerability, and efficacy of several doses of tropifexor (LJN452) in patients with non-alcoholic steatohepatitis (NASH).

Parts A and B of study CLJN452A2202 in NASH patients have investigated tropifexor at doses ranging from 10 to 90 μg daily for 12 weeks. Tropifexor exhibited a clear dose response for target engagement (FGF19) and biologic activity (GGT). ALT and hepatic fat fraction were reduced across all tropifexor doses (10, 30, 60 and 90 μg) compared to placebo. The study showed that Tropifexor was generally well tolerated up to 90 μg daily without safety signals. Results from the first two parts (A and B, study CLJN452A2202) demonstrated anti-inflammatory and anti-steatotic efficacy of 60 and 90 μg of tropifexor based on biomarkers, and favorable safety at Week 12.

In Part C, the effects of higher doses of tropifexor on biomarkers and histology will be evaluated over 48 weeks in patients with biopsy-proven NASH and fibrosis stages 2-3. In all, 152 patients (64% females) were randomized to receive placebo (N=51), tropifexor 140 μg (N=50) or tropifexor 200 μg (N=51) once daily. Prespecified endpoints assessed at week 12 included overall safety and changes in alanine aminotransferase (ALT), hepatic fat fraction (HFF), gamma glutamyl transferase (GGT), and body weight.

RESULTS: Pre-specified endpoints were met for tropifexor at a dose of 200 μg. Efficacy results are presented in Table 1.

TABLE 1
Least squares means of absolute changes in ALT, GGT,
and body weight, and relative change in HFF from
baseline to Week 12 estimated in repeated measures
or analysis of covariance models (full analysis set)

| | | Tropifexor | Tropifexor |
|---|---|---|---|
| | Placebo | 140 μg | 200 μg |
| Biomarkers | (N = 51) | (N = 50) | (N = 51) |
| ALT (U/L) | −8.9 (4.19) | −20.1 (4.57) | −23.6 (4.48) |
| | n = 49 | n = 41; | n = 39; |
| | | P = 0.058 | P = 0.013 |
| Relative change | −10.26 (4.21) | −16.99 (4.64) | −31.37 (4.30) |
| in HFF* (%) | n = 51 | n = 49; | n = 51; |
| | | P = 0.209 | P < 0.001 |
| GGT (U/L) | −2.5 (3.55) | −39.2 (3.70) | −40.9 (3.62) |
| | n = 49 | n = 44; | n = 46; |
| | | P < 0.001 | P < 0.001 |
| Body weight (kg) | −1.14 (0.36) | −2.46 (0.38) | −3.20 (0.37) |
| | n = 50 | n = 46; | n = 46; |
| | | P = 0.010 | P < 0.001 |

*Measured as magnetic resonance imaging-proton density fat fraction (MRI-PDFF).
Data are presented as LS mean change (SE) with 2-sided P values reported for statistical significance
ALT, alanine aminotransferase;
GGT, gamma glutamyl transferase;
HFF, hepatic fat fraction;
LS, least square;
SE, standard error;

Relative HFF reduction (without imputation for missing values) by ≥30% was achieved in 20%, 32%, and 64% of patients in the placebo, Tropifexor 140 μg, and Tropifexor 200 μg groups, respectively. The frequency of serious adverse events was low and comparable across groups. Among patients with pruritus, >60% in both Tropifexor groups and all in the placebo group experienced events with mild (Grade 1) severity. Treatment discontinuation rates due to pruritus were low (Tropifexor 140 μg: n=1 [2%]; Tropifexor 200 μg: n=3 [6%]; placebo: 0%). A dose-related increase in low density lipoprotein-cholesterol (LDL-C) was seen. None of the lipid changes led to treatment discontinuation or dose reduction.

In this prespecified interim analysis of Part C, higher doses of Tropifexor resulted in robust and dose-dependent decreases in ALT, HFF, and body weight with good safety and tolerability after 12 weeks of treatment.

### Example 3. Clinical Study for Efficacy, Safety, and Tolerability in Subjects with NASH and Fibrosis (Stage 2 or 3) as Per NASH CRN Histological Score

Primary objective: To demonstrate the efficacy of tropifexor as assessed by histologic improvement after 48 weeks of treatment in subjects with NASH and stage 2 or 3 fibrosis. Secondary objectives:
Improvement in fibrosis by at least one stage with no worsening of NASH after 48 weeks of treatment
Resolution of NASH with no worsening of fibrosis after 48 weeks of treatment
Improvement in fibrosis by at least one stage
Improvement in fibrosis by at least two stages with no worsening of NASH after 48 weeks of treatment
Reduction in body weight from baseline after 48 weeks of treatment
Change in liver fat content after 48 weeks of treatment
To determine the relationship of investigational treatment and markers of hepatic inflammation in NASH (ALT and AST)
To determine the relationship of investigational treatment and GGT, a marker of cholestasis

The study consists of 1) a screening period, 2) a treatment period starting from randomization on Day 1 and running to Week 48, and 3) a follow up period of 4 weeks after the last dose of study treatment. The screening period starts from the time of the signing of informed consent and continues for up to 8 weeks when all inclusion/exclusion criteria have been evaluated and all baseline assessments have been performed. The study duration from first dose of study medication is 52 weeks. The total duration of participation may be up to 60 weeks.

Subjects eligible for inclusion in this study must meet all of the following criteria:
Written informed consent must be obtained before any assessment is performed.
Male and female subjects 18 years or older (at the time of the screening visit)
Presence of NASH as demonstrated by the following during the screening period: NASH with fibrosis stage 2 or 3 confirmed by central reader's evaluation using NAFLD Activity Score (NAS) and NASH CRN criteria, of liver biopsy obtained no more than 6 months before randomization.
Able to communicate well with the investigator, to understand and comply with the requirements of the study

The planned duration of treatment is 48 weeks. Subjects may be discontinued from treatment earlier due to unacceptable tolerability, disease progression and/or at the discretion of the investigator or the subject.

Subjects (n=70) are assigned at baseline visit to tropifexor monotherapy Arm: tropifexor 140 μg, once daily. Subjects should take the medication in the evening following a meal and at about the same time each day, except at baseline and week 4 where the dose will be taken in the morning at the clinic instead of evening dose.

The efficacy assessments should be completed in the following recommended order:
MRI.
Liver function test: ALT, AST, GGT, total alkaline phosphatase (and isoenzymes if total alkaline phosphatase is >ULN, and 5′nucleotidase if either GGT or total alkaline phosphatase is >ULN during study participation), total bilirubin, and albumin will be assessed.
Protein measurements using SOMAscan.
Markers of liver fibrosis: originally called Fibrotest®/Fibrosure®. The following will be assessed: α2-macroglobulin, apolipoprotein A1, total bilirubin, haptoglobin, GGT, and ALT.
NAFLD fibrosis score: The following formula will be utilized for the calculation of the NAFLD fibrosis score: −1.675+0.037×age (years)+0.094×BMI (kg/m2)+1.13×IFG (increased fasted glucose)/diabetes (yes=1, no=0)+0.99×AST/ALT ratio−0.013×platelet (×109/l)−0.66×albumin (g/dl).
Fasting insulin and glucose: Blood samples will be collected for fasting insulin and glucose assessment.
Liver biopsy: Subjects must have histologic evidence of NASH and liver fibrosis stage 2 or 3 (NASH clinical research network (CRN) staging criteria) demonstrated on liver biopsy within 6 months prior to randomization.

In addition, a Transient Elastography (FibroScan®) can be done at screening/baseline and at the Week 12, 24 and, 48. Standard safety parameters and measures are collected including adverse events and serious adverse events according to definitions and process detailed in the protocol.

It is understood that the examples and embodiments described herein are for illustrative purposes only and that various modifications or changes in light thereof will be suggested to persons skilled in the art and are included within the spirit and purview of this application and scope of the appended claims. All publications, patents, and patent applications cited herein are hereby incorporated by reference for all purposes.

1-29. (canceled)

30. A method for treating a condition mediated by Farnesoid X receptor (FXR), comprising administering a therapeutically effective amount of an FXR agonist once daily in the evening to a subject in need thereof; and wherein said condition mediated by FXR is non-alcoholic fatty liver disease (NAFLD), non-alcoholic steatohepatitis (NASH), liver fibrosis or primary biliary cirrhosis (PBC).

31. The method of claim 30, wherein the FXR agonist is selected from tropifexor, obeticholic acid, nidufexor, cilofexor, TERN-101, EDP-305, PXL007, AGN242266 and MET409.

32. The method according to claim 31, wherein the FXR agonist is obeticholic acid.

33. The method according to claim 32, wherein obeticholic acid is administered at a daily dose of about 5 mg, of about 10 mg, of about 15 mg, of about 20 mg, of about 25 mg, of about 30 mg, of about 40 mg or of about 50 mg.

34. The method according to claim 30, wherein the FXR agonist is tropifexor.

35. The method according to claim 34, wherein tropifexor is administered at a daily dose of about 90 μg to about 250 μg, e.g. of about 140 μg to about 200 μg.

36. The method according to claim 34, wherein tropifexor is administered at a dose of about 90 μg/day, of about 140 μg/day, of about 150 μg/day, of about 160 μg/day, of about 170 μg/day, of about 180 μg/day, of about 190 μg/day, of about 200 μg/day, of about 210 μg/day, of about 220 μg/day, of about 230 μg/day, of about 240 μg/day or of about 250 μg/day.

37. The method according to claim 34, wherein tropifexor is administered at a daily dose of about 140 μg.

38. The method according to claim 30, wherein the FXR agonist is cilofexor.

39. The method according to claim 38, wherein cilofexor is administered at a daily dose of about 5 mg, of about 10 mg, of about 15 mg, of about 20 mg, of about 25 mg, of about 30 mg, of about 40 mg or of about 50 mg.

40. The method according to claim 38, wherein cilofexor is administered at a daily dose of about 30 mg, once daily.

41. The method according to claim 30, wherein said method reduces the risk of pruritus associated with administration of the FXR agonist.

42. The method according to claim 30, wherein said method reduces the risk of lipid abnormality associated with administration of the FXR agonist.

43. The method according to claim 30, wherein said method comprises resolution of steatohepatitis, improvement in liver fibrosis or a combination thereof.

44. The method of claim 30, comprising administering to said subject a combination of an ACC inhibitor and said FXR agonist.

45. The method according to claim 44, wherein the ACC inhibitor is selected from firsocostat, MK-4074 and PF-05221304.

46. The method according to claim 44, wherein the ACC inhibitor is firsocostat.

47. The method according to claim 46, wherein firsocostat is administered at a dose of about 5 mg, of about 10 mg, of about 15 mg, of about 20 mg, of about 25 mg, of about 30 mg, of about 40 mg or of about 50 mg.

48. The method according to claim 46, wherein firsocostat is administered at a daily dose of about 20 mg, once daily.

49. The method according to claim 44, wherein said method reduces drug-induced weight gain from said ACC inhibitor.
